(19) United States
(12) Patent Application Publication
(10) Pub. No.: US 2002/0006930 A1
(43) Pub. Date: Jan. 17, 2002

(54) Method for stimulating liver regeneration

(76) Inventor: Wilfred Wayne Lautt, Winnipeg (CA)

Correspondence Address: Kohn & Associates, Suite 410, 30500 Northwestern Highway, Farmington Hills, MI, 48334, US

(21) Appl. No.: 09/888,171

(22) Filed: Jun. 22, 2001

Related U.S. Application Data
(60) Provisional application No. 60/213,514, filed on Jun. 22, 2000.

(51) Int. Cl. A61K 31/519; A61K 31/517

(52) U.S. Cl. 514/258; 514/259

(57) ABSTRACT

There is provided an NO donor for use in regenerating the liver. Also provided is a pharmaceutical for liver regeneration including an effective amount of the chemical which promotes liver regeneration and a pharmaceutically acceptable carrier. Also provided is a method of stimulating liver regeneration by administering an effective amount of an NO donor or a chemical which stimulates cGMP production.

### CROSS-REFERENCE TO RELATED APPLICATIONS

This application claims the benefit of priority under 35 U.S.C. Section 119(e) of U.S. Provisional Patent Application No. 60/213,514, filed Jun. 22, 2001, which is incorporated herein by reference.

### BACKGROUND OF THE INVENTION

1. Technical Field

The present invention relates to liver regeneration. More specifically, the present invention relates to a pharmaceutical for use in stimulating liver regeneration.

2. Background Art

The Greek God, Zeus, used the regenerative capacity of the liver to torment Prometheus for the offense of passing the secret of fire to humans, by chaining him to a rock and having an eagle perform a daily partial hepatectomy (PHX) on him. The remarkable regenerative capacity of the liver has been studied for almost one hundred years and a systematic approach was made possible by the development of a standard operating procedure for producing a graded degree of surgical PHX (Higgins & Anderson, 1931). Using this model, numerous reports indicate that after a two-thirds PHX in rats, full restoration of liver mass occurs by one week with about 50% of restoration occurring at 48 hours. Although a great deal of information is known about the complex cascade of events that follows PHX, including growth factor and cytokine production and early gene responses followed by cell proliferation, (a recent series of reviews extensively covers these areas: Columbano & Shinozuka, 1996; Diehl & Rai, 1996; Fausto et al; 1995; Francavilla et al., 1994; Kren & Steer, 1996; Michalopoulos & DeFrances, 1997; Pistoi & Morello, 1996; Ponder, 1996; Taub, 1996; Thorgeirsson, 1996), it was not known if there is some primary triggering event that initiates the regeneration cascade. Michalopoulos & DeFrances (1977) concluded, in their review on liver regeneration, that “in analogy to studies of the big bang theory of the universe, research in liver regeneration still needs to sort out the earliest signals associated with triggering the origin of the regenerative response.”

It was recently shown that the initiating trigger is related to the instantaneous hemodynamic event that occurs upon removal of part of the liver. The portal blood supply accounts for approximately three-quarters of the blood flow to the liver and the liver does not control this blood flow which is simply the total of blood flows flowing out from the intestine, spleen, pancreas, stomach, and omentum. When a portion of the liver is removed, the total blood flow of the portal vein is forced through the remnant liver thus causing an increased ratio of blood flow to liver mass. The increase in portal flow velocity results in a shear stress induced release of nitric oxide (NO) in the liver (Macedo & Lautt, 1998). Shear stress is the frictional effect of blood flow acting on the delicate endothelial cells lining the interior lumen of blood vessels and is well recognized to cause NO release in a number of vascular beds.

The liver does not control portal venous blood flow, but must accept the entire outflow from other splanchnic organs. Portal venous blood flow has long been known to be an important factor for normal liver regeneration (1, 2). However, it has also been suggested that a humoral mechanism is responsible for triggering the liver regeneration cascade. This view began with studies using parabiotic animals, which suggested that a blood borne factor stimulated liver regeneration in an animal that had not undergone a regenerative stimulus (3). Candidates for the putative humoral factor include growth factors, such as hepatocyte growth (HGF), or cytokines, such as interleukin-1 (IL-1) or interleukin-6 (IL-6) (4). However, production of these growth factors and cytokines is not an immediate event. One event, occurring immediately after partial hepatectomy (PHX) or selective portal vein branch ligation (PVL), is a hemodynamic change. Ligation of the left portal venous branch supplying ⅔ of the liver or removal of ⅔ of liver tissue causes all the blood in the portal vein to flow through the remaining ⅓ of the liver. Considering the extreme rapidity with which the regeneration cascade commences, it is a reasonable assumption that some basic mechanism serves to trigger the process essentially at the moment of surgical removal of liver mass.

There has been little progress made in determining the mechanism by which portal flow influences liver mass restoration following PHX but the current consensus would appear to be that the portal flow per se is less important than trophic factors which can be carried in the portal blood. The first evidence supporting a role for blood-borne growth-promoting factors appearing after PHX was presented by Glinos & Gey (1952) who showed that cultured fibroblasts and hepatocytes proliferated under the stimulus of dilute serum from PHX animals and by Wenneker & Sussman (1951) who showed hepatic hypertrophy in a parabiotic twin that had not been subjected to PHX. By 1965, the “blood flow” theory had been discredited (Thompson & Clarke, 1965) although many of the early observations were never refuted nor has there seemed to be consideration that both views can be correct with blood flow effects serving as the trigger to unleash a cascade of growth regulating signals.

Although appearance of a number of proliferating factors (PF) in plasma is shown to occur within hours of initiation of the PHX surgery, a single initiating trigger has not been proposed or identified. It would therefore be useful to determine what triggers the liver regeneration cascade.

### SUMMARY OF THE INVENTION

According to the present invention, there is provided a NO donor for use in regenerating the liver. Also provided is a pharmaceutical for liver regeneration including an effective amount of the chemical which promotes liver regeneration and a pharmaceutically acceptable carrier. The method of stimulating liver regeneration by administering an effective amount of a liver stimulating compound is also provided.

In accordance with one aspect of the present invention, there is provided a method for increasing liver regeneration by the provision of exogenous or endogenous sources of NO. In accordance with another aspect of the present invention, there is provided a method for stimulation of liver regeneration by elevating intracellular cGMP either through application of chemicals that increase hepatic production of cGMP or by administration of compounds, such as phosphodiesterase inhibitors, that inhibit the intracellular destruction of cGMP.

### DESCRIPTION OF THE DRAWINGS

Other advantages of the present invention are readily appreciated as the same becomes better understood by reference to the following detailed description when considered in connection with the accompanying drawings wherein:

FIG. 1 is a graph showing PVP before and immediately after PHX and PVL;

FIG. 2 is a graph showing a liver weight distribution after PVL;

FIG. 3 is a graph showing production of PFs as an index of initiation of the liver regeneration;

FIG. 4 is a graph showing c-fos mRNA expression after PHX;

FIG. 5 is a graph showing hepatic c-fos mRNA expression following PVL;

FIG. 6 is a graph showing c-fos mRNA expression after ⅓ PHX and the addition of the phosphodiesterase antagonist; and

FIG. 7 is a graph showing the potentiation of c-fos mRNA expression as a result of the addition of an NO donor.

### DETAILED DESCRIPTION OF THE INVENTION

By way of background, the remarkable regenerative capacity of the liver has been studied for almost one hundred years (Fishback, 1929; Milne, 1909; Von Podwyssozki, 1886) and a systematic approach was made possible by the development of a standard operating procedure for producing a graded degree of surgical PHX (Higgins & Anderson, 1931). Using this model, numerous reports indicate that after a two-thirds PHX in rats, full restoration of liver mass occurs by one week with about 50% of restoration occurring at 48 hours. Michalopoulos & DeFrances (1997) concluded, in their review on liver regeneration, that “in analogy to studies of the big bang theory of the universe, research in liver regeneration still needs to sort out the earliest signals associated with triggering the origin of the regenerative response.” The data presented herein are consistent with the method of the present invention wherein PHX results in the entire portal flow being forced to flow through the remaining liver mass, thereby elevating shear stress and leading to generation of NO which serves as a trigger to initiate the cascade of events leading to restorative hyperplasia. The methods and compositions of the present invention serve to trigger this cascade.

In order for the hemodynamic consequence of PHX to serve as the trigger for the onset of the regeneration cascade, the hemodynamic event must lead to a rapid alteration of some chemical mediator. Based on the previous discovery of the mechanism of hepatic arterial blood flow regulation mediation through adenosine washout, the hypothesis that a decrease in adenosine as a result of the relative increase in portal flow through the remaining liver mass could serve as the trigger was tested. This hypothesis was disproven and applicant next turned to the possibility that the stimulus was shear stress-induced release of NO. It was recently demonstrated that shear stress in the portal circulation was capable of releasing NO that resulted in inhibition of vasoconstriction induced by sympathetic nerves or norepinephrine infusion (Macedo & Lautt, 1998). Using the pharmacological approaches from these vascular studies, the concept that shear stress-induced NO could serve as the trigger for the liver regeneration cascade was tested. The role of NO in the initiation was indirectly tested by administering L-NAME, a NO synthase antagonist, to block NO production in vivo. Three intravenous injections of L-NAME into two-thirds PHX rats at early time points after PHX blocked the appearance of PF in the plasma to the same level as seen in the sham PHX control group. Inhibition was maximum with a dose of 2.5 mg/kg and was not further inhibited by a dose of 5.0 mg/kg. The increase of plasma PF in PHX rats was completely prevented by blocking NO production. In order to further verify that NO played a role in the initiation of regeneration, L-arginine, the NO synthase substrate, were administered to reverse the L-NAME-induced blockade. L-Arginine significantly reversed the inhibition by L-NAME.

To determine whether NO served as a trigger to initiate a further cascade of events or whether it served as a direct proliferating stimulus on hepatocytes, the cultured hepatocytes were exposed to concentrations of L-NAME estimated to occur in the in vivo experiments. Blockade of NO production in vitro produced a positive proliferative stimulus indicating that the direct effect of NO on hepatocytes was antiproliferative and that the effect in vivo could not be due to a direct stimulation of NO on hepatocytes but rather must be due to NO activating a further cascade.

The question of whether blockade of PF appearance in the plasma also resulted in inhibition of in vivo liver regeneration has proven somewhat more difficult to evaluate. The extent of liver regeneration can be assessed from a number of parameters, the most direct being the restoration of liver weight following PHX. The control animals showed a normal degree of restoration of liver weight 48 hours post-PHX (47±3.8%) regeneration. The low dose of L-NAME did not significantly inhibit regeneration but the high dose (two administrations of 2.5 mg/kg followed by 30 mg/kg/24 hours subcutaneous infusion) did show a significant suppression of regeneration (33±4.7%). The results therefore support the concept of the present invention that the ability to inhibit regeneration is compatible with a triggering role for NO.

Generally, the present invention provides methods and compositions for stimulating the regeneration of the liver. More specifically, the present invention provides an NO donor for use in regenerating the liver.

The compositions and pharmaceuticals of the present invention are intended to include, but are not limited to, the use of classes of drugs that either elevate intrahepatic NO or intrahepatic cGMP to stimulate liver regeneration (hyperplasia). These classes of drugs can include the following drugs: S-Nitrosothiol Compounds, 3-Morpholinosydnonimine (SIN-1) (5 mg/kg) S-nitroso-N-acetyl-D,L-penacillamine (SNAP) (5 mg/kg), Sodium nitroprusside GEA 3162, O2-vinyl l-(pyrrolidin-l-yl)diazem-l-ium-1,2-diolate, Diazenium diolates, Glyceryl trinitrate, Pentaerythrityl tetranitrate, Isosorbide dinitrate, Isosorbide 5-mononitrate, Cysteine, N-acetyl-cysteine, Thiosalicyclic acid, S-nitrosoalbumin, S-nitrosoglutathione, Moldisomine, Nonoates, including spermine nonoate and diethylatmine nonoate, and L-arginine. The compositions can include any exogenous or endogenous source of NO. This allows the NO to either be imported into the body or it can allow a chemical to be added which stimulates the production of NO within the body. Additionally, the compositions can include any compounds which increase intracellular cGMP either through the application of chemicals which increase hepatic production of cGMP or by the administration of compounds which inhibit intracellular destruction of cGMP. Examples of such compounds include, but are not limited to, phosphodiesterase inhibitors, which include, but are not limited to, Zaprinast (10 mg/kg), Sildenafil, E-4021, MBCQ (4-[[3,4-(methylenedioxy)benzyl]amino]-6-chloroquinazoline), T-1032, SKF-96231, 1,3-dimethyl-6-(2-propoxy-5-methanesulfonylamidophenyl)-pyrazolo[3,4-d]pyrimidin-4-(5H)-one, ONO-1505 (4-[2-(2-hydroxyethoxy)ethylamino]-2-(1-H-imidazol-l-yl)-6-methoxyquin azoline methanesulphonate), UK-122764, and DMPPO (1,3 dimethyl-6-(2-propoxy-5-methane sulphonylamidophenyl)-pyrazolo[3,4-d]pyrimidin-4-(5H)-one).

Also provided is a pharmaceutical for liver regeneration including an effective amount of the chemical which promotes liver regeneration and a pharmaceutically acceptable carrier. The chemical is a chemical as set forth previously. The pharmaceutically acceptable carrier is a carrier known to one of skill in the art to be compatible with the present use.

The method of stimulating liver regeneration by administering an effective amount of a liver stimulating compound is also provided. The stimulation can occur through an increase in the amount of cGMP available either through a reduction in the intracellular destruction of cGMP or by increasing the production of cGMP. Alternatively, the stimulation can occur by increasing the exogenous or endogenous source of NO.

The above discussion provides a factual basis for the use of method of and composition for stimulating liver regeneration. The methods used with and the utility of the present invention is shown by the following non-limiting examples and accompanying figures.

### EXAMPLES

Methods

General methods in molecular biology: Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In-situ (in-cell) PCR in combination with Flow Cytometry can be used for detection of cells containing specific DNA and mRNA sequences (Testoni et al, 1996, Blood 87:3822.)

Delivery of Gene Products/Therapeutics (Compound)

The compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound or as pharmaceutically acceptable salt and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

It is noted that humans are treated generally longer than the mice or other experimental animals exemplified herein which treatment has a length proportional to the length of the disease process and drug effectiveness. The doses can be single doses or multiple doses over a period of several days, but single doses are preferred. The treatment generally has a length proportional to the length of the disease process and drug effectiveness and the patient species being treated.

When administering the compound of the present invention parenterally, it is generally formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it is desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the compound utilized in the present invention can be administered orally to the patient. Conventional methods such as administering the compounds in tablets, suspensions, solutions, emulsions, capsules, powders, syrups and the like are usable. Known techniques which deliver it orally or intravenously and retain the biological activity are preferred.

In one embodiment, the compound of the present invention can be administered initially by intravenous injection to bring blood levels to a suitable level. The patient's levels are then maintained by an oral dosage form, although other forms of administration, dependent upon the patient's condition and as indicated above, can be used. The quantity to be administered can vary for the patient being treated and can vary from about 100 ng/kg of body weight to 100 mg/kg of body weight per day and preferably can be from 10 mg/kg to 10 mg/kg per day.

### EXAMPLE 1

The question of whether blockade of PF appearance in the plasma also resulted in inhibition of in vivo liver regeneration has proven somewhat more difficult to evaluate. The extent of liver regeneration can be assessed from a number of parameters, the most direct being the restoration of liver weight following PHX. The control animals showed a normal degree of restoration of liver weight 48 hours post-PHX (47±3.8%) regeneration. The low dose of L-NAME did not significantly inhibit regeneration but the high dose (two administrations of 2.5 mg/kg followed by 30 mg/kg/24 hours subcutaneous infusion) did show a significant suppression of regeneration (33±4.7%). Unfortunately, the liver regeneration studies are made complex by the well known observation that NO synthase blockade can potentiate the toxic reaction to inflammatory conditions. It has been suggested that NO blockade disrupts the balance between constrictor and dilator agents and leads to stellate cell contraction and blood flow heterogeneity in the liver in conditions such as ischemia-reperfusion and endotoxemia (Clemens et al., 1997). This can have been a factor in these studies where 6 of 14 rats died in the high dose group between 36 and 44 hours. These animals had only 18±4.7% regeneration. The ability to inhibit regeneration is compatible with a triggering role for NO.

Other Evidence for Hemodynamio Control of Regeneration Triggering

It has long been recognized that diversion or portal blood flow into the vena cava results in atrophy of the liver (Mann, 1940). Early studies in dogs (Mann, 1940) showed that if the excess portal flow/liver mass was diverted from the portal vein into the vena cava, liver regeneration was minimal. Selective ligation of the left branch of the portal vein, which supplies blood to two-thirds of the liver, results in atrophy of the liver lobes deprived of portal blood and hypertrophy of the perfused lobes (Mann, 1940; Um et al., 1998).

Theoretical calculations and experimental results indicate that an increase in portal perfusion pressure reflects an increase in shear stress and results in a release of NO (Macedo & Lautt, 1998). (This is a very useful index that can be extensively used in the hemodynamic studies described later). It is known that a two-thirds PHX results in a maintained portal blood flow thus resulting in an elevated flow through the remaining one-third of the liver and a subsequent increase in portal pressure (Kahn et al., 1984; Lee et al., 1987; Rabinovici & Weiner, 1963; Rice et al., 1977) and, therefore, an increase in shear stress.

In the selective portal vein ligation (PVL) model, the hemodynamic effect is similar to what is seen in a two-thirds PHX animal. Portal blood flow was shown not to decrease so that the total portal flow was forced through the unligated portal branches which led to an increase in portal pressure. The unligated lobes underwent hypertrophy which became stabilized after seven days when the portal pressure, and therefore shear stress, also stabilized (Um et al., 1994). A recent study (Yamakado et al., 1997) reported that portal vein embolization induces atrophy of the embolized hepatic parenchyma and hypertrophy of the unembolized liver, but only if the extent of portal vein embolization is sufficient to cause an elevation in portal venous pressure and, therefore, in shear stress. These models are consistent with the foundation of the present invention that it is the increase in shear stress that leads to NO release and triggering of the regeneration cascade.

A Second Hemodynamic initiator Model

When the two-thirds PHX model is used, two-thirds of the liver is surgically removed thus forcing all of the portal blood flow through the remaining one-third of the liver which results in increased shear stress and release of NO. It was determined that by occluding the portal venous branches that supply the same two-thirds of the liver as is normally surgically removed, the entire portal blood flow is directed through one-third of the liver. This was a critical hypothesis to verify that the blood flow played a major role. When animals were allowed to recover from the selective portal vein ligation (PVL), the liver weight of the unligated lobes (equivalent to the remnant lobes after PHX) progressively increased to become stabilized within one week (Schoen et al., 2000). This showed that the increased portal flow to the unligated lobes was sufficient stimulus to lead to hypertrophy of the liver.

It was further demonstrated that the index of increased shear stress in the liver, elevated portal venous pressure, was similar in the two-thirds PHX and two-thirds PVL animals (Schoen et al., 2000). This demonstrates that the hemodynamic impact of both experimental maneuvers results in similar hemodynamic events in the liver.

If the hemodynamic event is acting as a trigger of the liver regeneration cascade, the appearance of proliferating factors four hours after the PVL should be similar to the appearance of proliferating factors in the plasma four hours after PHX. When proliferating factors were analyzed using the in vitro assay previously described, the elevation in proliferating factors was similar with both models thus demonstrating that the PVL hemodynamic stimulus was capable of stimulating elevated levels of proliferating factors in the plasma. Thus both events appear to be able to initiate shear stress-induced release of NO with a resultant generation of proliferating factors that is able to be completely prevent by blockade of NO production.

Nitric Oxide as the Common Stimulus for Liver Regeneration Both for the PHX Model and Following Liver Damage

The PHX or the PVL model is a useful tool that directly relates to situations where liver hypertrophy is desired following resection of portions of the liver or following liver transplantations using small livers. However, most clinically relevant liver regeneration is required following liver damage induced by viral or chemical insults. There is also evidence that NO serves as the triggers in these situations. Barriault et al., (1977) have suggested that activation of NO production by Kupffer cells exposed to a number of toxins is associated with a proliferative wave in hepatic cells in the absence of morphological or biochemical evidence of hepatocyte damage. They point out that this wave of hepatocyte proliferation coincided with protection against the toxin. A parallel between proliferation of hepatocytes and protection against phalloidin had been observed earlier. It was reported that PHX rats and newborn animals were resistant to phalloidin (Chap et al., 1985; Fleckenstein et al., 1996; Hendrickse & Triger, 1993). In reviewing the literature, these studies were made complex by the administration of toxins, and by the fact that excess NO production in the liver can actually potentiate toxic effects. Barriault et al., (1997) conclude that “altogether, the available information supports the view that phalloidin hepatoprotection by agents modulating Kupffer cell activity can be explained by their mitotic effect on hepatocytes.” Their conclusions are compatible with local tissue damage resulting in release of NO, probably from Kupffer cells, which would then lead to liver regeneration secondary to NO triggering the regenerative cascade was shown in the PHX model. Although speculative, the interpretation of these studies is that they are consistent with the possibility that NO could serve as the primary trigger for liver regeneration regardless of whether its release is subsequent to either an inflammatory reaction, as in the liver injury models, or to a hemodynamic effect (shear stress), as in the PHX and PVL models.

### EXAMPLE 2

c-Fos as an Early Indicator of Regeneration Triggering

In the previous studies the indication of the hemodynamic events serving as a trigger for liver regeneration was determined from responses measured well after the initiation of the stimulus either using PVL or PHX. The ability to suppress liver regeneration after PHX was determined from animals that had been recovered for 48 hours. Much closer to the initiating trigger, the appearance of proliferating factors in the plasma was studied at four hours after PHX or PVL. Changes in expression of several immediate- and delayed-early genes occur following PHX (Haber et al., 1992). Among the immediate early genes, c-fos mRNA expression is increased, peaking at 15 minutes after PHX (Moser et al., 1997). C-fos mRNA expression increases proportional to the degree of PHX performed (Moser et al., 197). In addition, c-fos mRNA expression increases in response to shear stress (Hsieh et al., 1993; Ranjan & Diamond, 1993). For these reasons, c-fos mRNA expression was selected as an index of initiation of the liver regeneration cascade closer to the triggering event. For this study both the PVL and PHX models were used.

Fifteen minutes after PVL or PHX, the livers were removed and c-fos mRNA expression was determined. c-Fos mRNA levels were significantly elevated to a similar extent in both models but were not elevated in the ligated lobes of the PVL model thereby demonstrating that c-Fos mRNA elevation was not a non-specific stress stimulus. When animals were pretreated with an inhibitor of NO synthase (L-NAME) designed to block NO production, c-fos mRNA expression was not significantly stimulated. When an exogenous NO donor (SIN-1) was administered following L-NAME but prior to PHX or PVL, the activation of c-fos was restored to normal levels.

Additional data reports an NO donor (SIN-1) was capable of elevating c-Fos in animals that had not been subjected to PHX or that had undergone some degree of prior stimulation as a result of a reduced degree of PHX, in this case a one-third PHX. Thus, in situations where the regenerative stimulus to the liver is not maximal, provision of exogenous NO is capable of activating the trigger that leads to hyperplasia. This shows that appropriate provision of NO to the liver as the target organ is capable of stimulating or potentiating liver cell replication. Because the first event resulting from elevation of NO is the increase of cGMP, the ability of drugs that elevate cGMP levels (through inhibition of the phosphodiesterase that normally destroys cGMP) were further tested to stimulate c-fos mRNA expression. Administration of a phosphodiesterase antagonist is anticipated to produce the same index of liver regeneration as was produced by PHX, selective PVL, and provision of NO using the NO donor.

### EXAMPLE 3

The trigger of the liver regeneration cascade is currently unknown and has been the subject of debate. It was hypothesized that, following ⅔ partial hepatectomy (PHX), and increase in the blood flow-to-liver mass ratio results in shear stress-induced nitric oxide (NO) release, which triggers the liver regeneration cascade. Portal venous pressure (PVP), reflecting shear stress in the liver, increased on the same extent following PHX, suggesting similar amounts of shear stress in both models. Two indices of the initiation of the liver regeneration cascade were used: proliferative factor (PF) activity in blood four hours and hepatic c-fos mRNA expression 15 minutes after PHX or PVL. PF activity and c-fos mRNA expression were increased to similar extents after PHX and PVL, suggesting a similar stimulus in both models. PF activity and c-fos mRNA expression were inhibited by administration of the NOS antagonist, L-NAME and the NO donor, SIN-1, reversed the inhibition in both models. These results provide support for the hypothesis that a hemodynamic change results in increased shear stress in the liver causing generation of NO, which then triggers the liver regeneration cascade.

### EXAMPLE 4

Methods

Animals

Male Sprague-Dawley rats, 250 g, were fed standard laboratory chow of libitum until the day before the experiment, when they were fasted for 8 hours and fed for two hours prior to experimentation.

Surgical Preparation

Partial Hepatectomy (PHX)

Animals were divided into four groups: sham procedures, PHX, PHX+L-NAME (Sigma), a Nos antagonist, and PHX+L-NAME+3morpholinosydnonimine (SIN-1, Alexis Corp.), a NO donor. Animals were anesthetized by sodium pentobarbital (6.5 mg/100 g. i.p; MTC Pharmaceuticals). Anesthesia was maintained using a continuous i.v. infusion (Harvard infusion pump, model 2720) of sodium pentobarbital (1.08 mg/ml saline, 1.0 ml/100 g/hr) via a cannula inserted into the femoral vein (PE50), Intramedic Clay Adams Brand, Becton-Dickinson). All surgical procedures were performed between the 10:00 am and 2:00 pm. Arterial blood pressure was monitored via a cannula (PE50, Intramedic Clay Adams Brand, Becton-Dickson) inserted into the femoral artery. To facilitate breathing a tracheotomy was performed using a polyethylene tube (PE240, Intramedic Clay Adams Brand, Becton-Dickinson) inserted into the trachea. Body temperature was maintained at 37.5±0.5° C. by the use of a rectal thermometer (Hanna Instruments) and a heated surgical table (Harvard Apparatus). Laparotomy was performed and a 24 G cather (Optiva™, Johnson and Johnson Medical Inc.) was inserted into the portal vein. The rat was then allowed to stabilize for 30 minutes.

A 0.5 ml bolus of L-NAME (5.0 mg/kg), or saline was infused intravenously over two minutes. Ten minutes later, PHX (resection of the left lateral and median lobes as described by Higgins and Anderson (20) or gentle manipulation of the liver lobes was performed. Resected lobes were litigated using black braided surgical silk (size 0, Ethicon) and excised. In the PHX+L-NAME+SIN-1 group a 0.1 ml bolus of SIN-1 (5.0 mg/kg) was infused intraportally over one minute, immediately before PHX. Fifteen minutes after PHX, or sham manipulation, the remaining liver was removed, slashed and blotted to remove blood, and immediately frozen on dry ice. Portal venous pressure (PVP) was measured, using an R-611 SensorMedics Dynograph Recorder, immediately before and after PHX.

For additional experiments involving PHX+L-NAME, the surgical procedure was exactly the same at that for the ⅔ PHX described above, except that the remaining liver as removed at 30, 45 or 60 minutes after PHX.

Selective Portal Vein Branch Ligation (PVL)

Rats were divided into four groups: sham PVL, PVL, PVL+L-NAME and PVL+L-NAME+SIN-1. Surgical procedures were the same as described for PHX except that liver lobes were not ligated and resected. Rather, the left branch of the portal vein was carefully isolated and resected. The left branch of the portal vein was carefully isolated from the surrounding hepatic artery, hepatic nerves, bile duct and lymphatics. A thread was slipped around the left branch of the portal vein and the animal was allowed to recover for one hour. Following the recovery period, saline or L-NAME was administered as described for the PHX group, and the PVL+L-NAME+SIN-1 group received SIN-1 as indicated above. The left branch of the portal vein was ligated and liver tissue was removed 15 minutes after PVL, slashed and blotted, and immediately frozen in dry ice for RNA isolation. For rats in the sham PVL group, the left branch of the portal vein was isolated, but not ligated and the liver lobes were gently manipulated in a similar manner to the experimental groups. The extent of PVL was verified by injection India ink into the portal vein of PVL and sham operated animals immediately before removal of the liver. Only livers with complete ligation of portal venous blood supply to the left lateral and median lobes were included in the experimental groups. PVP was measured immediately before and after PVL, as described for PHX surgery.

Liver Weight Restoration after PVL

Surgery was conducted under sterile conditions between 10:00 am and 2:00 pm. Rats were anesthetized using sodium pentobarbital (6.5 mg/100 g body weight, i.p.) Fifteen minutes before PVL, 0.5 ml saline was injected i.v. into the tail vein. PVL surgery was the same as describe above, except that no tracheotomy or cannulations were performed. The incision was sutured using Dexon II, 3-0, C-6 sutures (26 mm, Davis & Geck). Banamine, an analgesic (2.5 mg/kg) was injected s.c. The rats were than allowed to recover. Post surgical care was given, with the rats allowed free access to food and water, and the cages maintained at 37° C. using heating pads (Solid State T/pump, Gaymar).

At 0, 2, 4 and 7 days after PVL, the rat was anesthetized using sodium pentobarbital (as above), the abdomen was opened and blood was drawn from the heart to facilitate drainage from the liver. The portal vein was punctured with a 26 G needle, and 1 ml of India ink was injected to verify ligated and nonligated lobes. The ligated and nonligated lobes were removed, slashed and blotted, and weighted separately. The percent liver lobe hypertrophy or atrophy was expressed as the percent weight of the lobe to the total liver weight: ligated (or nonligated) lobe weight/total liver weight×100%.

In vitro Bioassay for PE Production

The production of proliferative factors (PFs) was determined as previously described (21) . Briefly, PHX or PVL was performed as described above. Four hours after PHX or PVL, the diaphragm was cut and a blood sample drawn (5 to 10 mp per rat) from the right ventricle of the heart using a 21 G 1.5-inch needle and a 10 ml sterile syringe. In a tissue culture hood, the blood was transferred to a sterile centrifuge tube, centrifuged at 2500 g for 20 minutes, and plasma stored at −20° C. The collagenase perfusion and hepatocyte isolation method was modified from Seglen (22), and has been previously described (21). Male Sprahue-Dawley rats (250 g) were anesthetized using sodium pentobarbital (6.5 mg/100 g, i.p.) and livers were perfused according to Seglen (22). Isolated cells were seeded (1×10⁵ cells/ml, 2 ml per well) in six-well culture plates and allowed to attach to the plate overnight at 37° C. The next day, unattached cells were washed out of the wells and the starting viable cell number was counted from three randomly selected wells. Trypsin-EDTA (Gibco, BRL) was used to isolate attached hepatocytes from the plate and the hepatocytes were counted using a hemocytometer (Fisher Scientific). Fresh medium and plasma samples from PHX rats (10% final concentration) were added to the rest of the wells (three wells per sample). The medium, containing plasma, was changed at 24 hours. The attached viable cells were harvested at 48 hours. and the final count of viable hepatocytes was determined. The PF levels were expressed as net cell proliferation (viable cell count per well at 48 hours minus starting cell count per well).

RNA Isolation and Northern Blot Analysis

Total RNA was isolated by a method modified from Chirgwin et al. (23). Liver tissue was homogenized (Brinkman, Polytron) in lithium chloride/urea (3M/6M; Sima). The homogenized was centrifuged at 25000 rpm (Brinkman L8-70M Ultracentrifuge) for 20 minutes at 4° C. Total RNA was extracted using phenol/chloroform (Fisher Biotech/Sigma) and allowed to precipitate overnight in sodium acetate/ethanol (Sigma) at −70° C. The samples were then centrifuged at 15000 rpm (Beckman Microfuge E) for 15 minutes at 4° C. The supernatant was decanted and the pellet was resuspended in sterile distilled water. The concentration of the RNA was then determined using a spectrophotometer (Ultrospec2000, Pharamacia Biotech). Twenty micrograms of total RNA were loaded onto a 1% agarose (Gibco BRL), 2.2 mol/L formaldehyde (Sigma) 1×MOPS denaturing gel. mRNA were separated by electrophoresis at 100 mV for 90 minutes and then transferred overnight to a GT-Zeta nylon membrane (Bio-Rad) by capillary action. The RNA was crosslinked to the membrane usi9ng a UV crosslinker (UV GS Genelinker, Bio-Rad). The membranes were prehybridized at 42° C. for 3 hours in prehybridization buffer consisting of 5×SSC, 7% SDS (Sigma), sheared denatured salmon sperm DNA (100 μg/ml, Gibco BRL) and 5×Denhardt's solution (Sigma). c-fos mRNA was detected using a 2.1 kb cDNA probe (kindly provided by Dr. C Steer, university of Minnesota, Minneapolis, Minn.). The cDNA probe was labeled by the random prime method using a commercial kit (Gibco BRL) with α-dCTP³²P (ICN Isotopes) and added to the prehybridization buffer. The membranes were hybridized overnight at 42° C., and washed using solutions of 2×SSC and 0.1% SDS, 0.5×SSC and 0.1% SDS, and 0.1×SSC and 0.1% SDS at 65° C. Autoradiography was performed using Kodak XAR film at −70° C. using an intensifying screen. The density of the c-fos mRNA band was quantitated using an HP scanner and the density of the c-fos mRNA band analyzed with the NIH Image 1.6 Densitometric Analysis Program (National Institute of Health, Bethesda, Md. c-fos mRNA expression is reported relative to 18S rRNA.

Statistics

Results were analyzed using one-way ANOVA followed by Turkey's test to determine differences between the groups. A p value less than 0.05 was deemed significant.

Results

Portal Venous Pressure After PHX and PVL

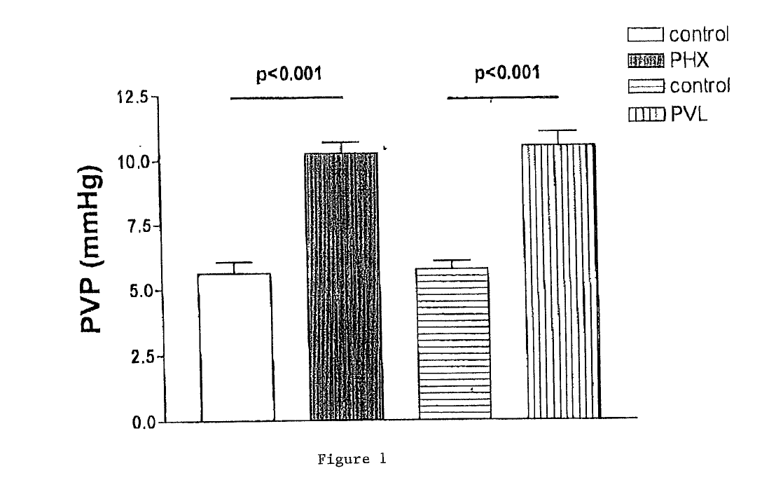

The similarity of hepatic hemodynamics in the PHX and PVL models was evaluated by comparing changes in PVP before and immediately following both PHX and PVL. The PVP increased immediately following PHX (10.25±0.43 mmHg. vs. 5.63±0.43 g, n=4, p<0.001) FIG. 1). A similar increase in PVP was observed after PVL 10.50±0.54 mmHg vs. 5.75±0.32 mmHg, n=4, p<0.001). The PVP was not different before PHX or PVL (FIG. 1), showing that both PHX and PVL cause similar increases in PVP. Thus, the shear stress load in the portal circulation is judged to be similar with ⅔ PHX and selective ⅔ PVL.

Liver Weight Redistribution Following PVL

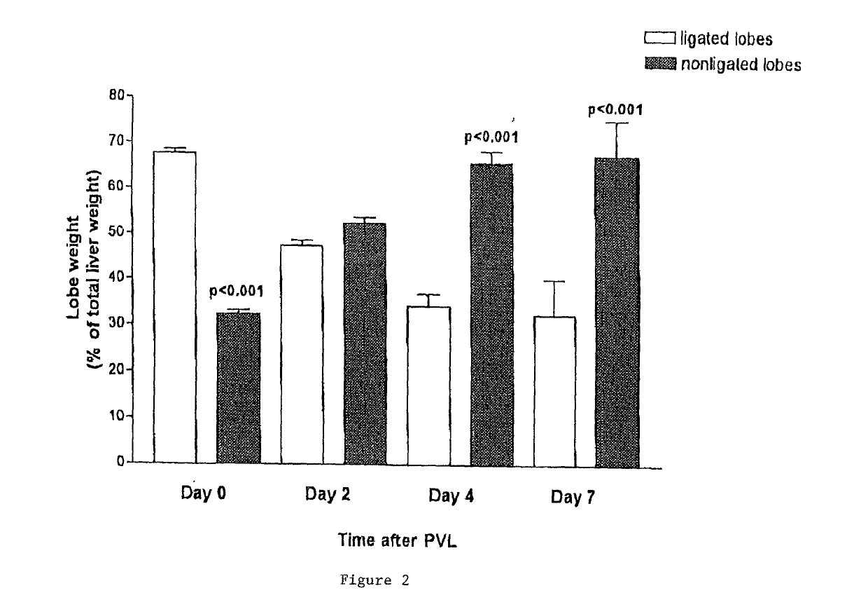

Ligation of the left branch of the portal vein deprives ⅔ of the liver of portal venous blood flow. PVL, a hemodynamic model of compensatory hyperplasia was used to evaluate the effect of a change in portal venous blood flow on liver mass. Liver weight redistribution was evaluated at 0, 2, 4 and 7 days after PVL. Immediately following PVL (day 0), the weight of the ligated lobes (67.75±0.93%, n=15) was significantly higher than the weight of the nonligated lobes (32.28±0.93%, n=15, p<0.001) (FIG. 2). However, by 48 hours after PVL, there was no difference between the weight of ligated and nonligated lobes (47.44±1.25% vs. 52.57±1.25%, n=4, N.S.) (FIG. 2). On days 4 and 7 after PVL, the weight of the ligated lobes was significantly less than that of the nonligated lobes (34.21±2.66% vs. 65.79±2.66%, n=5, p<0.001; and 32.45±7.72% vs. 67.54±7.72, n=5, p<0.001, respectively) (FIG. 2). These results suggest that an inverse relationship is present between the amount of liver mass hypertrophy after PVL in the nonligated lobes and the amount of atrophy in the ligated lobes. The data are consistent with the hypothesis that the hemodynamic stimulus of PVL, intended to mimic the hemodynamic changes that occur following PHX, leads to compensatory hepatic hyperplasia, similar to hypertrophy occurring after PHX.

PF Production After PHX and PVL

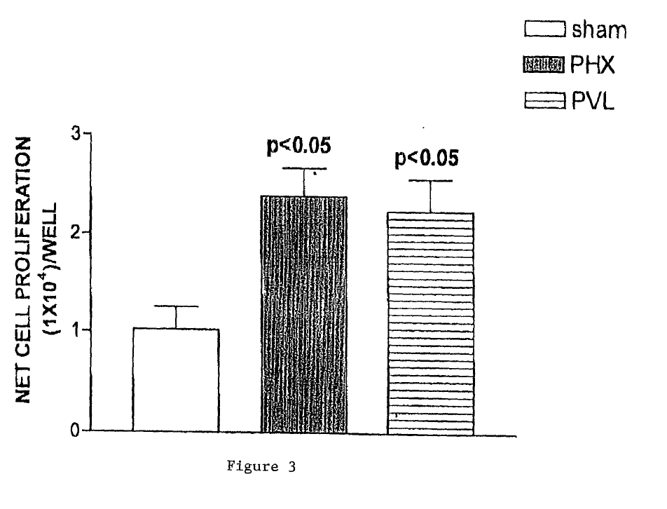

As indicated above, similar hemodynamic conditions seem to be present in both the PHX and PVL models, and thus both were used to test the hypothesis that shear stress-induced NO, released in response to a hemodynamic change, triggers the liver regeneration cascade. As a test of the hypothesis, a previously evaluated index of initiation of the liver regeneration cascade, production of PFs after PHX, was compared to that after PVL. PF production was found to peak 4 hours after PHX (Wang and Lautt, 1997a), and this timepoint was used to determine PF production after PHX and PVL. Production of PFs four hours after PHX (n=9, 2.40±0.29) and PVL (n=5, 2.24±0.34) was greater than that after sham (n=12) (1.02±0.23, p<0.01 and p<0.05, respectively) (FIG. 3). However, PF production was not different between PHX and PVL groups (FIG. 3), suggesting similar stimulation of PF production in both models.

c-Fos mRNA Expression After PHX

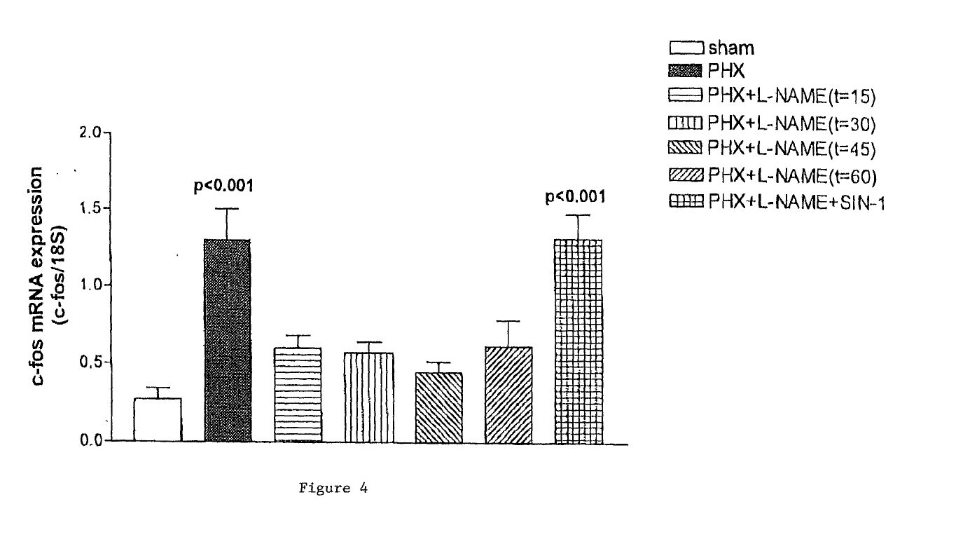

The hypothesis was further tested using an additional index of initiation of the liver regeneration cascade, c-fos mRNA expression, closer to the triggering event, c-Fos mRNA expression increased 15 minutes after PHX (1.30±0.20; n=5) compared to sham operated rats (0.27±0.07, n=7, p<0.001) (FIG. 4). This increase was blocked by administration of L-NAME (n=6; 5.0 mg/kg i.v.) to sham levels (0.60±0.08, N.S. from sham), and reversed by the NO donor, SIN-1 (n=5; 5.0 mg/kgi. p.v.) (1.32±0.16,p<0.001 vs. sham; FIG. 4). These results suggest that NO is involved in the increase in c-fos mRNA expression after PHX.

As shown in FIG. 4, c-fos mRNA expression is reduced to sham levels by inhibition of NOS by the nonspecific NOS inhibitor, L-NAME. To test the hypothesis that L-NAME inhibits, and does not simply delay, the peak of c-fos mRNA expression, L-NAME (5.0 mg/kg i.v.) was administered to rats prior to PHX and the liver was removed at 30, 45 and 60 minutes after PHX (n=5 each group). c-Fos mRNA expression was then evaluated and compared to that at 15 minutes after PHX. There was no difference in c-fos mRNA expression at any of these four time points after PHX (FIG. 4), suggesting that NOS blockade inhibits c-fos mRNA expression after PHX.

c-Fos mRNA Expression After PVI

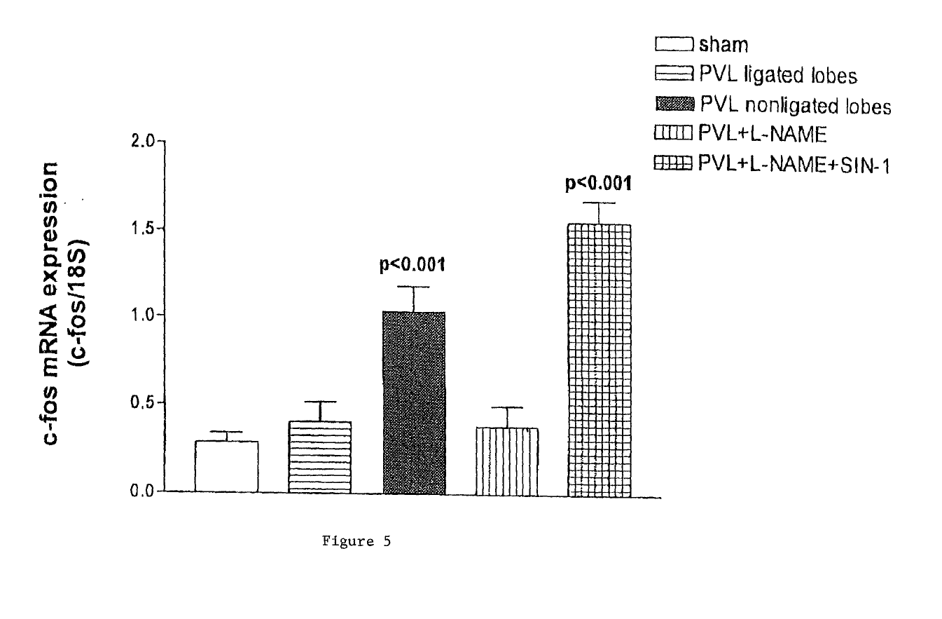

To further evaluate the hemodynamic involvement in triggering the liver regeneration cascade, c-Fos mRNA expression was evaluated 15 minutes after PVL. c-Fos mRNA expression increased significantly after PVL (1.04±0.15, n=8) compared to sham (0.29±0.05, n=8, p<0.01) (FIG. 5). This increase in c-fos mRNA expression was inhibited by L-NAME (n=4; 5.0 mg/kg i.v.) (0.38±0.12, N.S. from sham), and the inhibition was reversed by the NO donor SIN-1 (n=8; 5.0 mg/kg i.p.v.) (1.56±0.13, p<0.001 compared to sham). These results show that c-Fos mRNA expression increases in response to hemodynamic changes in hepatic blood flow. Also, the results provide further support for the hypothesis that a hemodynamic event causes an increase in shear stress and the release of NO, which then triggers the liver regeneration cascade.

c-Fos mRNA expression was evaluated in the ligated (left lateral or median) lobes 15 minutes after PVL. There was no change in c-Fos mRNA expression from sham levels in the ligated lobes of the liver following PVL (0.40±0.11, n=5, N.S.) (FIG. 5). These results show that c-Fos mRNA was selectively expressed in the nonligated lobes, but not in the ligated lobes, following PVL.

Discussion

Results of the experiments support the hypothesis that an increase in the blood flow-to-liver mass ratio results in an increase in shear stress and the release of NO, which triggers the liver regeneration cascade. Portal venous pressure increased after PHX, a model of liver regeneration, and after PVL, a hemodynamic model of hyperplasia similar to PHX, suggesting an increase in shear stress in both models. Liver weight redistribution following PVL was half complete after 48 hours, continuing to completion by 7 days. This is similar to PHX, suggesting similarly between the two models. The similarity of the two models was further tested using a previously described in vitro bioassay (21) to detect PF production at 4 hours after PHX and PVL. PHX results in the appearance of a variety of proliferating factors (PFs) that can be assayed as a generic group by the ability to stimulate hepatocyte proliferation in cultured hepatocytes, when exposed to plasma from rats that underwent PHX. PF production, assayed using the bioassay, which is sufficiently sensitive to differentiate between PF production after sham, ⅓ PHX and ⅔ PHX (21), was not different following PHX or PVL, suggesting a similar stimulus for PF production following both PHX and PVL.

To further test the hypothesis, an index of initiation of the liver regeneration cascade, which was closer to the actual triggering event, was employed. c-Fos mRNA expression increased 15 minutes after PHX, as previously described (17). The increase in c-Fos mRNA expression was inhibited by L-NAME, a NOS antagonist, and the inhibition was reversed by administration of SIN-1, a NO donor. The inhibition of c-Fos mRNA expression by L-NAME is not merely a delay in the peak of c-Fos mRNA expression, but an inhibition. Also, using the PVL model, the hemodynamic involvement in c-Fos mRNA expression was evaluated. As after PHX, c-Fos mRNA expression increased 15 minutes after PVL. This increase was blocked by L-NAME and the blockade was reversed by SIN-1, which provides further support for the hypothesis that an increase in the blood flow-to-liver mass ratio causes a shear stress and the release of NO, which triggers the liver regeneration cascade.

More specifically, FIG. 1 shows PVP before and immediately after PHX and PVL. PHX and PVL were performed as described in the Methods. The data represent means±S.E. PVP increased to the same extent after both PHX and PVL, suggesting that a similar amount of shear stress is induced in the liver by both models.

Additionally, FIG. 2 shows the liver weight distribution after PVL. The liver lobe weight is expressed as the percent of the total liver weight: ligated (or nonligated) lobe weight/total liver weight×100%. The bars represent mean±S.E. The weight of the nonligated lobes increased and that of the ligated lobes decreased, suggesting a reciprocal relationship between hypertrophy and atrophy in the PVL model.

FIG. 3 shows the production of PFs, as an index of initiation of the liver regeneration cascade, after PHX and PVL. Plasma samples were prepared from rats four hours after sham, PHX or PVL, and tested using an in vitro hepatocyte bioassay. The bars represent mean±S.E. Production of PFs four hours after PVL was not different from that after PHX, suggesting that a similar stimulus for initiation of the liver regeneration cascade is present after both PHX and PVL.

Further, FIG. 4 shows c-Fos mRNA expression after PHX. Hepatic c-fos mRNA expression increased 15 minutes after PHX compared to sham operated rats. The increase was blocked by administration of L-NAME, and reversed by the NO donor, SIN-1. These results suggest that NO is involved in the increase in c-fos mRNA expression after PHX. Also, L-NAME inhibits c-fos mRNA expression in the liver after PHX. c-Fos mRNA expression is reduced to sham levels by inhibition of NOS by the nonspecific NOS inhibitor, L-NAME at 15, 30, 45 and 60 minutes after PHX. There was no difference in c-fos mRNA expression at any of these four time points after PHX, suggesting that NOS blockade inhibits, rather than delays, c-fos mRNA expression after PHX.

FIG. 5 shows the hepatic c-fos mRNA expression following PVL. c-FOS mRNA expression increased significantly in the nonligated lobes 15 minutes after PVL compared to sham. The increases in c-fos mRNA expression were inhibited by L-NAME, and the inhibition was reversed by the NO donor SIN-1. These results show that c-fos mRNA expression increases in response to hemodynamic changes in hepatic blood flow. Also, c-fos mRNA expression did not increase in the ligated lobes after PVL, suggesting that c-fos mRNA is selectively expressed in the nonligated lobes in response to a hemodynamic change rather than surgical trauma, and is a good index for the initiation of the liver regeneration cascade.

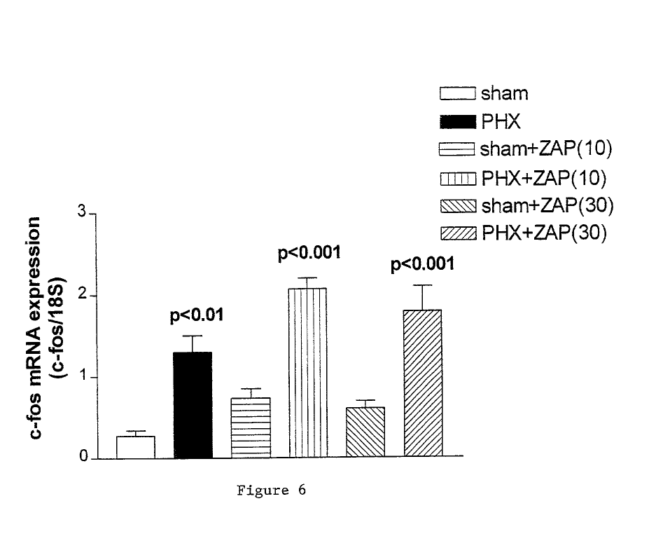

Additionally, FIG. 6 shows that the NO donor, SNAP (5 mg/kg), potentiates c-fos mRNA expression, an index of initiation of the liver regeneration cascade. The NO donor, SNAP, potentiates c-fos mRNA expression after PHX but has no effect on c-fos mRNA expression in sham operated livers. These results support the hypothesis that NO potentiates the liver regeneration cascade.

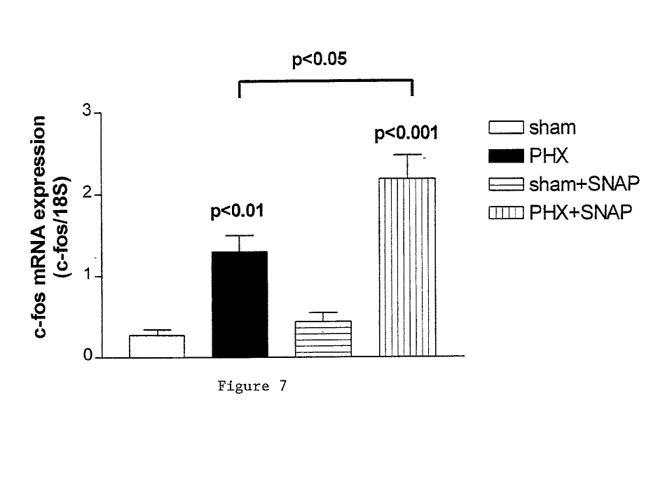

Finally, FIG. 7 shows the c-fos mRNA expression after ⅔ PHX and the phosphodiesterase antagonist, Zaprinast (ZAP; 10 and 30 mg/kg). C-Fos mRNA expression after PHX in potentiated by Zaprinast, but not after sham procedures. Zaprinast potentiates c-fos mRNA expression through inhibition of cGMP breakdown, thereby increasing the effect of NO signaling, when the liver regeneration cascade has been stimulated, but not in the normal liver. These results are consistent with the hypothesis that NO potentiates the liver regeneration cascade and acts through the intracellular mediator cGMP.

### EXAMPLE 5

The experiment was aimed to develop and optimize a hepatocyte primary culture bioassay to detect proliferative factors (PF) in the plasma or serum of partial hepatectomized (PHX) rats, which could serve as an index of the initiation of liver regeneration cascade.

The bioassay detects the presence of PF by measuring hepatocyte proliferation through a directly counting cell number increases over the culture period using a hemacytometer. The procedure of liver perfusion and hepatocyte purification was adopted from Seglen (1976 Methods Cell Biol. 13, 29) using Sprague Dawley rats. The purified hepatocytes (0.80% viability, 0.95% parenchymal cells) were seeded into 6-well culture plates and allowed to attach to the plate overnight at 37° C. The unattached cells were then washed out and the starting cell count was determined from 3 randomly picked wells in the culture after trypsin digestion. Sera collected from ⅔ PHX rats at 1-6 hours post PHX was added to the culture. With a medium change at 24 hours, the final cell counting was performed at 48 hours. The net cell proliferation was expressed as the difference between the counts at 48 hours and starting at hour (0 hr).

The optimized assay conditions (100.000.00 cells/ml plating concentration, 10% serum, 48 hr culture) were able to detect an increase of PF in PHX rat serum between 1-4 hours after PHX, with a peak activity at 4 hours (unpooled serum n=5/time point, pooled n=5/point). The assay has a high sensitivity to detect PF in ⅓ PHX rat serum (n=7), which was not detectable using the conventional DNA synthesis method.

This methodology allows for identification for the presence of PF in PHX rat serum, which is a useful tool to assess the initiation of the liver regeneration cascade.

### EXAMPLE 6

Following partial hepatectomy (PHX), a cascade of events (growth factor elevation, immediate-early gene responses) leading to rapid liver restoration is triggered. The initial step triggering the regeneration cascade is highly controversial. It was hypothesized that the hemodynamic change (↑blood flow/liver mass ratio) occurring immediately upon PHX serves as the initial trigger and that this ratio change alters the concentration of flow-dependent factor(s) that leads to production of proliferative factors (PF) that represent the onset of the regeneration cascade.

To test the hypotheses, an in vivo hepatocyte primary culture bioassay was developed to detect PF in the plasma of PHX rats as an index of the initial trigger. The PHX surgery was performed according to the conventional method. Liver weight was also measured from the post PHX recovered rats to directly assess liver regeneration in vivo. The level of flow-dependent factors (e.g. adenosine (Ado), NO) was pharmacologically manipulated to test their roles in triggering the liver regeneration cascade.

An early generation of PF from 1-4 hours after PHX, peaking at 4 hours, was detected in the plasma of ⅔ PHX rats (n=5 rats/point) using the primary culture (n=11 cultures). Ado had no significant effects on PF generation (6=n rats, 6 cultures). L-NAME (NO synthase antagonist) was able to both block 4 hours peak PF generation in the plasma (n=11 rats, 7 cultures) and inhibit the liver weight regeneration in vivo (n=14 rats). The 36-44 hours post surgery survival rate was only about 50% in L-NAME group (n=14) versus 100% in PHX (n=9) and L-NAME-no-PHX (n=6) control groups. L-arginine, No synthase substrate, reversed the L-NAME inhibitory effect in vivo (n=7).

The data is consistent with the hypothesis that the hemodynamic change (↑flow/mass ratio) upon PHX causes shear stress-dependent release of NO that initiates the cascade of liver regeneration.

### EXAMPLE 7

Recent experiments in the laboratory support the hypothesis that nitric oxide (NO), released secondary to increased vascular shear stress, triggers the liver regeneration cascade. Blockade of NO synthase by N-nitro-Larginine methyl ester (L-NAME), inhibited the increase in c-fos mRNA expression, an index of initiation of the liver regeneration cascade, after both ⅔ partial hepatectomy (PHX) and selective portal vein branch ligation (PVL). The inhibition was reversed by 3-morpholinosydnonimine (SIN-1), a NO donor. The c-fos proto-oncogene was chosen as an index because it was previously shown that c-fos mRNA expression increases after PHX, peaking at 15 minutes. Also, c-fos nRNA expression increases in response to shear stress. Thus, it was hypothesized that c-fos mRNA expression and liver regeneration, could be potentiated by NO. To test this hypothesis. a submaximal regenerative stimulus, ⅓ PHX, was used. Rats were divided into four groups: ⅓ PHX, ⅓ PHX+SIN-1, sham or ±SIN-1. Liver tissue was removed 15 minutes after PHX, and total hepatic mRNA was extracted and analyzed by northern blotting, with c-fos mRNA expressed relative to 18S rRNA. c-fos mRNA expression in livers of rats after ⅓ PHX did not increase significantly from those of sham rats (0.35+/−0.05 vs. 0.27+/−0.07, N.S.). However, administration of SIN-1 to rats, which underwent ⅓ PHX, caused an increase in c-fos mRNA expression compared to sham and ⅓ PHX (0.88+/−0.10, p<0.01 and p<0.05, respectively). This shows that c-fos mRNA expression following PHX can be potentiated by NO. In addition, administration of SIN-1 to sham animals increased c-fos mRNA expression compared to sham only (0.83+/−0.09, p<0.01), showing that NO can also potentiate c-fos mRNA expression in livers of normal rats. These results support the hypothesis that NO potentiates c-fos mRNA expression in the regenerating rat liver, and suggest that NO is involved in potentiation of the liver regeneration cascade.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

References

Arnold, W. P., Mittal, C. K., Katsuki, S., Murad, F. 1977. Nitric oxide activates guanylate cyclase and increases guanosine 3′:5′-cyclic monophosphate levels in various tissue preparations. Proc. Natl. Acad. Sci. USA 74: 3203-3207.

Barriault, C., Yousef, I. M., and Tuchweber, B. 1997. Hepatoprotection by agents which modulate macrophage activity may be mediated by their mitogenic properties. Drug Metab. Rev. 29: 397-411.

Brzozowski, T., Konturek, S. J., Drozdowicz, D., Dembinski, A., and Stachura, J. 1995. Healing of chronic gastric ulcerations by L-arginine: role of nitric oxide, prostaglandins, gastrin and polyamines. Digestion 56: 463-471.

Bucher, N. L. R. 1963. Regeneration of mammalian liver. Int. Rev. Cytol. 15: 245-300.

Chaps, Z., Ishida, T., Chou, J., Lewis, R., Hartley, C., Entman, M., and Field, J. B. 1985. Effects of atropine and gastric inhibitory polypeptide on hepatic glucose uptake and insulin extraction in conscious dogs. J. Clin. Invest. 76: 1174-1181.

Clemens, M. G., Bauer, M., Pannen, B. H. J., Bauer, I., and Zhang, J. X. 1997. Remodeling of hepatic microvascular responsiveness after ischemia/reperfusion. Shock 8: 80-85.

Columbano, A. and Shinozuka, H. 1996. Liver regeneration versus direct hyperplasia. FASEB J. 10: 1118-1128.

Counts, W. B. and Flanim, W. G. 1966. An artifact associated with the incorporation of thymidine into DNA preparations. Biochim. Biophys. Acta 114: 628-630.

Diehl, A. M. and Rai, R. M. 1996. Regulation of signal transduction during liver regeneration. FASEB J. 10: 215-227.

Fausto, N., Laird, A. D., and Webber, E. M. 1995. Role of growth factors and cytokines in hepatic regeneration. FASEB J. 9: 1527-1536.

Fishback, F. C. 1929. A morphologic study of regeneration of the liver after partial removal. Arch. Path. 7: 955-977.

Fleckenstein, J. F., Frank, S. M., and Thuluvath, P. J. 1996. Presence of autonomic neuropathy is a poor prognostic indicator in patients with advanced liver disease. Hepatology 23: 471-475.

Francavilla, A., Hagiya, M., Porter, K. A., Polimeno, L., Ihara, I., and Starzl, T. E. 1994. Augmenter of liver regeneration: its place in the universe of hepatic growth factors. Hepatology 20: 747-757.

Glinos, A. D. and Gey, G. O. 1952. Humoral factors involved in the induction of liver regeneration in the rat. Proc. Soc. Exp. Biol. Med. 80: 421-425.

Grindlay, J. H. and Bollman, J. L. 1952. Regeneration of the liver in the dog after partial hepatectomy. Surg. Gynec. Obstet. 94: 491-496.

Grisham, J. W. 1960. Inhibitory effect of tritiated thymidine on regeneration of the liver in the young rat. Proc. Soc. Exp. Biol. Med. 105: 555-558.

Haber, B. A., Mohn, K. L., Diamond, R. H., and Taub, R. 1992. Induction patterns of 70 genes during nine days after hepatectomy define the temporal course of liver regeneration. J. Clin. Invest. 91: 1319-1326.

Hendrickse, M. T. and Triger, D. R. 1993. Autonomic dysfunction in chronic liver disease. Clin. Auton. Res. 3: 227-231.

Higgins, G. M. and Anderson, R. M. 1931. Experimental pathology of the liver: I. Restoration of the liver of the white rat following partial surgical removal. Arch. Pathol. 12: 186-202.

Hsieh, H. J., Li, N. Q., and Frangos, J. A. 1993. Pulsatile and steady flow induces c-fos expression in human endothelial cells. J. Cell Physiol. 154: 143-151.

Kahn, D., van Hoorn-Hickman, R., and Terblanche, J. 1984. Liver blood flow after partial hepatectomy in the pig. J. Surg. Res. 37: 290-294.

Kren, B. T. and Steer. C. J. 1996. Posttranscriptional regulation of gene expression in liver regeneration: role of mRNA stability. FASEB J. 10: 559-573.

Lee, S. S., Hadengue, A., Girod, C., Braillon, A., and Lebrec, D. 1987. Reduction of intrahepatic vascular space in the pathogenesis of portal hypertension in vitro and in vivo studies in the rat. Gastroenterology 93: 157-161.

Macedo, M. P. and Lautt, W. W. 1998. Shear-induced modulation of vasoconstriction in the hepatic artery and portal vein by nitric oxide. Am. J. Physiol. 274: G253-G260.

Magrimat, G. C., Mason, S. N., Sahmi, P. J., and Weinberg, J. B. 1992. Nitric oxide modulation of human leukaemia cell differentation: induction of tumor necrosis factor on RNA by nitric oxide. Clin. Res. 40: 297A.

Mann, F. C. 1940. The portal circulation and restoration of the liver after partial removal. Surgery 8: 225-238.

Michalopoulos, G. K. and DeFrances, M. C. 1997. Liver regeneration. Science 276: 60-66.

Milne, L. S. 1909. The histology of liver tissue regeneration. J. Path. Bact. 13: 127-160.

Morley, C. G. D. and Kindgon, H. S. 1972. Use of ³H-thymidine for measurement of DNA synthesis in rat liver: a warning. Anal. Biochem. 45: 298-305.

Moser, M. J., Gong, Y., Zhang, M. N., Johnston, J., Lipschitz, J., Kneteman, N. M., and Minuk, G. Y. 1997. Immediate-early protooncogene expression and liver function following varying extents of partial hepatectomy in the rat. Hepatology 26(4): 567A.

Pistoi, S. and Morello, D. 1996. Prometheus' myth revisited: transgenic mice as a powerful tool to study liver regeneration. FASEB J. 10: 819-828.

Ponder, K. P. 1996. Analysis of liver development, regeneration, and carcinogenesis by genetic marking studies. FASEB J. 10: 673-684.

Rabinovici, N. and Wiener, E. 1963. Hemodynamic changes in the hepatectomized liver of the rat and their relationship to regeneration. J. Surg. Res. 3: 3-8.

Ranjan, V. and Diamond, S. L. 1993. Fluid shear stress induces synthesis and nuclear localization of c-fos in cultured human endothelial cells. Biochem. Biophys. Res. Commun. 196: 79-84.

Rice, G. C., Leiberman, D. P., Mathie, R. T., Ryan, C. J., Harper, A. M., and Blumgart, L. H. 1977. Liver tissue blood flow measured by ⁸⁶Kr clearance in the anesthetized rat before and after partial hepatectomy. Br. J. Exp. Pathol. 58: 243-250.

Salvemini, D. 1997. Regulation of cyclooxygenase enzymes by nitric oxide. Cell. Mol. Life Sci. 53: 576-582.

Schoen, J. M., Wang, H. H., Minuk, G. Y., and Lautt, W. W. 2001. Shear stress-induced nitric oxide release triggers the liver regeneration cascade. Nitric Oxide (in press).

Shiratori, Y., Hougo, S., Hikiba, Y., Ohmura, K., Nagura, T., Okano, K., Kamii, K., Tanake, T., Komatsu, Y., Ochiai, T., Tsubouchi, H., and Omata, M. 1996. Role of macrophages in regeneration of liver. Dig. Dis. Sci. 41: 1939-1946.

Soma, M., Izumi, Y., Watanabe, Y., and Kanmatsuse. K. 1996. A nitric oxide synthesis inhibitor decreased prostaglandin production in rat mesenteric vasculature. Prostaglandins 51: 225-232.

Taub, R. 1996. Transcriptional control of liver regeneration. FASEB J. 10: 413-427.

Thomson, R. Y. and Clarke, A. M. 1965. Role of portal blood supply in liver regeneration. Nature 208: 392-393.

Thorgeirsson, S. S. 1996. Hepatic stem cells in liver regeneration. FASEB J. 10: 1249-1256.

Um, S., Nishida, O., Tokubayashi, M., Kimura, F., Takimoto, Y., Yoshioka, H., Inque, R., and Kita, T. 1994. Hemodynamic changes after ligation of a major branch of the portal vein in rats: comparison with rats with portal vein constriction. Hepatology 19: 202-209.

Von Podwyssozki Jr., W. 1886. Experimentalle untersuchungen uber die regeneration der drusengewebe. Beitr. Z. Path. Anat. U.Z Allg. Path. 1: 259.

Wang, H. and Lautt, W. W. 1997a. Does nitric oxide (NO) trigger liver regeneration? Proc. West. Pharmacol. Soc. 40: 17-18.

Wang, H. H. and Lautt, W. W. 1997b. Hepatocyte primary culture bioassay—a simplified tool to assess the initiation of the liver regeneration cascade. J. Pharmacol. Toxicol. Meth. 38: 141-150.

Webber, E. M., Godowski, P. J., and Fausto, N. 1994. In vivo response of hepatocyte to growth factors requires an initial priming stimulus. Hepatology 14: 489-497.

Wenneker, A. S. and Sussman, N. 1951. Regeneration of liver tissue following partial hepatectomy in parabiotic rats. Proc. Soc. Exp. Biol. Med. 76: 683-686.

Wu, K. K. 1995. Inducible cyclooxygenase and nitric oxide synthase. Adv. Pharmacol. 33: 179-207.

Yamakado, K., Takeda, K., Matsumura, K., Nakatsuka, A., Hirano, T., Kato, N., Sakuma, H., Nakagawa, T., and Kawarada, Y. 1997. Regeneration of the un-embolized liver parenchyma following portal vein embolization. J. Hepatol. 27: 871-880:

What is claimed is:

1. A NO donor for use in regenerating the liver.

2. A pharmaceutical for liver regeneration comprising:
an effective amount of a chemical which promotes liver regeneration and a pharmaceutically acceptable carrier.

3. The pharmaceutical according to claim 2, wherein said chemical stimulates NOS.

4. The pharmaceutical according to claim 2, wherein said chemical stimulates the generation of cGMP.

5. The pharmaceutical according to claim 2, wherein said chemical is a phosphodiesterase inhibitor.

6. The pharmaceutical according to claim 5 wherein said phosphodiesterase inhibitor is selected from the group consisting essentially of Zaprinast, Sildenafil, E-4021, MBCQ (4-[[3,4-(methylenedioxy)benzyl]amino]-6-chloroquinazoline), T-1032, SKF-96231, 1,3-dimethyl-6-(2-propoxy-5-methanesulfonylamidophenyl)-pyrazolo[3,4-d]pyrimidin-4-(5H)-one, ONO-1505 (4-[2-(2-hydroxyethoxy)ethylamino]-2-(1H-imidazol-l-yl)-6-methoxyquin azoline methanesulphonate), UK-122764, and DMPPO (1,3 dimethyl-6-(2-propoxy-5-methane sulphonylamidophenyl)-pyrazolo [3,4-d]pyrimidin-4-(5H)-one).

7. A method of stimulating liver regeneration by administering an effective amount of a liver stimulating compound.

8. The method according to claim 7, wherein said administering step includes administering a NO donor.

9. The method according to claim 7, wherein said administering step includes administering a compound to stimulate cGMP formation.

10. A method of inducing liver regeneration by elevating intracellular cGMP.

11. The method according to claim 10, wherein said elevating step includes decreasing the rate of cGMP destruction.

12. The method according to claim 10, wherein said elevating step includes increasing the rate of cGMP synthesis.

13. The method according to claim 10, wherein said elevating step includes exogenously adding cGMP.

14. The method according to claim 10, wherein said elevating step includes exogenously adding means for increasing cGMP.
